(19) United States
(12) Patent Application Publication
(10) Pub. No.: US 2012/0310134 A1
(43) Pub. Date: Dec. 6, 2012

(54) LIGAMENT WRAP FOR HORSES

(76) Inventor: Kathleen Smith, N. Tonawanda, NY (US)

(21) Appl. No.: 13/153,443

(22) Filed: Jun. 5, 2011

(51) Int. Cl. A61F 13/06

(52) U.S. Cl. 602/62

(57) ABSTRACT

The present invention is a horse ligament wrap with a perimeter that includes a front wrap made of washable quilted fabric, a back wrap made of washable quilted fabric and a piece of vinyl fabric with a front side and a back side that is placed between the front wrap and the back wrap The horse ligament wrap also includes a securing tab that secures the horse ligament wrap around a horse's injured area and a plurality of stitching securing the front wrap, the back wrap and the vinyl fabric together forming the ligament wrap. The horse ligament wrap is also used in combination with a liniment to work as a sweat.

### TECHNICAL FIELD & BACKGROUND

Currently there are limited options for effective animal bandages and horse leg wraps. Traditional horse leg wraps utilized to prevent tendon swelling, often unravel after application, thus causing the animal discomfort in addition to improper shielding of a bruised area.

The present invention generally relates to a ligament wrap. More specifically, the invention is a ligament wrap for horses.

It is an object of the invention to provide a ligament wrap for a horse that does not require a plastic wrap.

It is an object of the invention to provide a ligament wrap for a horse that is one piece.

It is an object of the invention to provide a ligament wrap for a horse that is machine washable.

What is really needed is a ligament wrap for a horse that does not require a plastic wrap that is one piece and that is machine washable.

### BRIEF DESCRIPTION OF THE DRAWINGS

The present invention will be described by way of exemplary embodiments, but not limitations, illustrated in the accompanying drawings in which like references denote similar elements, and in which:

FIG. 1A illustrates a front perspective view of a front wrap and a back wrap of a ligament wrap, in accordance with one embodiment of the present invention.

FIG. 1B illustrates a front perspective view of a piece of vinyl fabric of a ligament wrap, in accordance with one embodiment of the present invention.

FIG. 1C illustrates a front perspective view of a piece of vinyl fabric, a front wrap and a back wrap of a ligament wrap, in accordance with one embodiment of the present invention.

FIG. 1D illustrates a front perspective view of a piece of vinyl fabric, a front wrap, a back wrap and a securing tab of a ligament wrap, in accordance with one embodiment of the present invention.

### DETAILED DESCRIPTION OF ILLUSTRATIVE EMBODIMENTS

Various aspects of the illustrative embodiments will be described using terms commonly employed by those skilled in the art to convey the substance of their work to others skilled in the art. However, it will be apparent to those skilled in the art that the present invention may be practiced with only some of the described aspects. For purposes of explanation, specific numbers, materials and configurations are set forth in order to provide a thorough understanding of the illustrative embodiments. However, it will be apparent to one skilled in the art that the present invention may be practiced without the specific details. In other instances, well-known features are omitted or simplified in order not to obscure the illustrative embodiments.

Various operations will be described as multiple discrete operations, in turn, in a manner that is most helpful in understanding the present invention. However, the order of description should not be construed as to imply that these operations are necessarily order dependent. In particular, these operations need not be performed in the order of presentation.

The phrase “in one embodiment” is utilized repeatedly. The phrase generally does not refer to the same embodiment, however, it may. The terms “comprising”, “having” and “including” are synonymous, unless the context dictates otherwise.

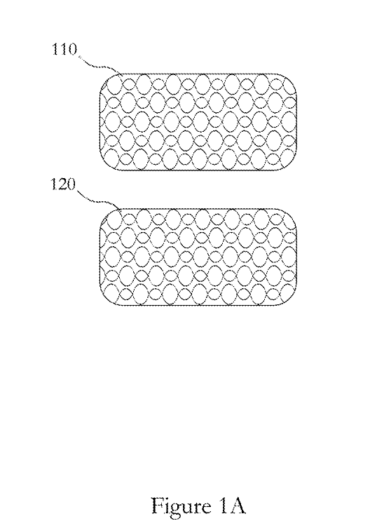

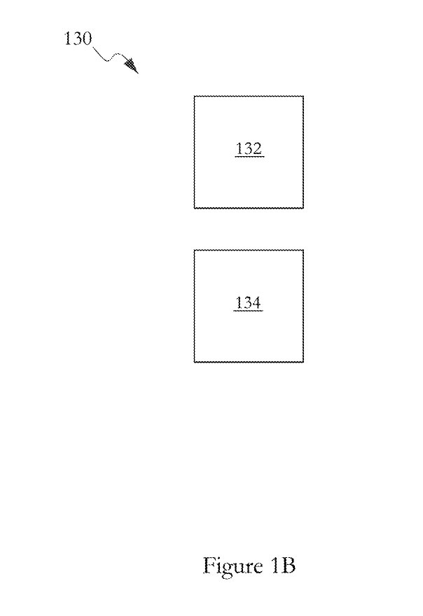

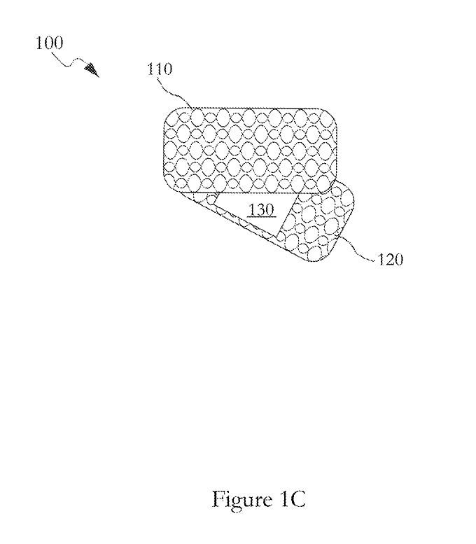

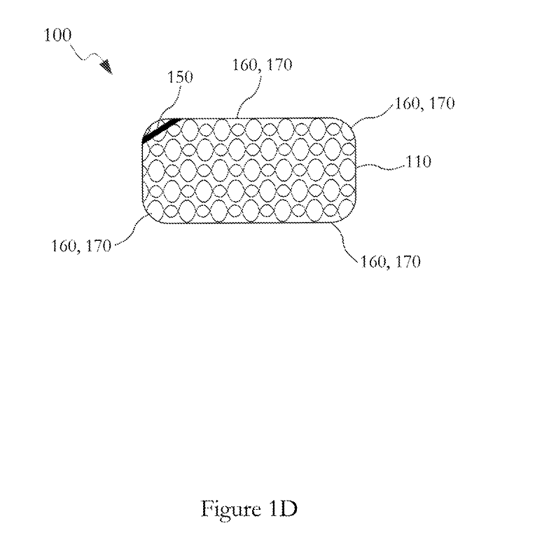

FIG. 1A illustrates a front perspective view of a front wrap 110 and a back wrap 120 of a ligament wrap 100, in accordance with one embodiment of the present invention. The ligament wrap 100 includes a front wrap 110 and a back wrap 120. The front wrap 110 is oval-shaped, relatively lightweight and is made of washable quilted fabric. The back wrap 120 is oval-shaped, relatively lightweight and is made of washable quilted fabric although the front wrap 110 and the back wrap 120 can be made of any suitable material. The size of the front wrap 110 and the back wrap 120 are both approximately 26 inches by 13 inches, although the front wrap 110 and back wrap 120 dimensions can be any suitable dimensions. The ligament wrap 100 can be used in combination with liniment L to allow the ligament wrap 100 to work as a sweat applied to a horse's leg.

FIG. 1B illustrates a front perspective view of a piece of vinyl fabric 130 of a ligament wrap 100, in accordance with one embodiment of the present invention. The ligament wrap 100 includes a piece of vinyl fabric 130 with a front side 132 and a back side 134. The vinyl fabric 130 can also be made of any other suitable material. The approximate dimensions of the vinyl fabric 130 are 15 inches by 12.5 inches, although the vinyl fabric 130 can be other suitable dimensions.

FIG. 1C illustrates a front perspective view of a piece of vinyl fabric 130, a front wrap 110 and a back wrap 120 of a ligament wrap 100, in accordance with one embodiment of the present invention. The vinyl fabric 130 is placed between the front wrap 110 and the back wrap 120 as part of forming the ligament wrap 100.

FIG. 1D illustrates a front perspective view of a piece of vinyl fabric 130, a front wrap 110, a back wrap 120 and a securing tab 150 of a ligament wrap 100, in accordance with one embodiment of the present invention. The ligament wrap 100 includes a securing tab 150 and a plurality of stitching 160 securing the front wrap 110, the vinyl fabric 130 and back wrap 120 of the ligament wrap 100. The securing tab 150 is approximately 5 inches long and is formed at an angle in a corner 102 of the ligament wrap 100. The securing tab 150 is a fastener that provides a steady and snug fit around a horse's injured area, such as a tendon, as the ligament wrap 100 is wrapped around the injured area of a horse. The stitching 160 is provided around the perimeter 170 of the ligament wrap 100 securing the front wrap 110, the back wrap 120 and vinyl fabric 130 together forming the ligament wrap 100.

The ligament wrap is a fabric support wrap that protects an injured horse from further physical injury. The ligament wrap is made of an external quilted cloth with an embedded single layer vinyl cover and a cornered 5 inch tab, which provides a steady and snug fit around a horse's tendon. This all-in-one lightweight swathe encases a horses' ligament without the necessity of additional support items, thereby greatly assisting in expediting the animal's healing process. The ligament wrap can be marketed in various sizes and color patterns to appeal to diverse consumers. The ligament wrap can be utilized while liniment is on a horse's leg, allowing the ligament wrap to work as a sweat.

The ligament wrap offers stable hands or horse caretakers a cost-saving leg wrap alternative. The feature of a 26″ inch surface area bandage, that is machine washable and adaptive to long term wear, can be a valuable animal medical device within the field of stallion management.

While the present invention has been related in terms of the foregoing embodiments, those skilled in the art will recognize that the invention is not limited to the embodiments described. The present invention can be practiced with modification and alteration within the spirit and scope of the appended claims. Thus, the description is to be regarded as illustrative instead of restrictive on the present invention.

1. A horse ligament wrap with a perimeter, comprising:
a front wrap made of washable quilted fabric;
a back wrap made of washable quilted fabric;
a piece of vinyl fabric with a front side and a back side that is placed between said front wrap and said back wrap;
a securing tab that secures said horse ligament wrap around a horse's injured area; and
a plurality of stitching provided around said perimeter of said ligament wrap securing said front wrap, said back wrap and said vinyl fabric together forming said ligament wrap.

2. The horse ligament wrap according to claim 1, wherein said front wrap is oval shaped.

3. The horse ligament wrap according to claim 1, wherein said back wrap is oval shaped.

4. The horse ligament wrap according to claim 1, wherein said securing tab is disposed on a corner of said horse ligament wrap.

5. The horse ligament wrap according to claim 1, wherein said horse ligament wrap is used in combination with a liniment to work as a sweat.

6. A horse ligament wrap with a perimeter, comprising:
a front wrap made of washable quilted fabric;
a back wrap made of washable quilted fabric;
a piece of vinyl fabric with a front side and a back side that is placed between said front wrap and said back wrap;
a securing tab that secures said horse ligament wrap around a horse's injured area; and
a plurality of stitching securing said front wrap, said back wrap and said vinyl fabric together forming said ligament wrap.

7. The horse ligament wrap according to claim 6, wherein said front wrap is oval shaped.

8. The horse ligament wrap according to claim 6, wherein said back wrap is oval shaped.

9. The horse ligament wrap according to claim 6, wherein said securing tab is disposed on a corner of said horse ligament wrap.

10. The horse ligament wrap according to claim 6, wherein said securing tab is colored and is approximately 5 inches long and said vinyl fabric is approximately 15 inches wide and 12.5 inches long.

11. The horse ligament wrap according to claim 6, wherein said stitching is provided around said perimeter of the ligament wrap.

12. The horse ligament wrap according to claim 6, wherein said horse ligament wrap is used in combination with a liniment to work as a sweat.

13. A horse ligament wrap with a perimeter, comprising:
a front wrap made of washable quilted fabric;
a back wrap made of washable quilted fabric;
a piece of vinyl fabric with a front side and a back side that is placed between said front wrap and said back wrap;
a securing tab that secures said horse ligament wrap around a horse's injured area; and
a plurality of stitching securing said front wrap, said back wrap and said vinyl fabric together forming said ligament wrap.

14. The horse ligament wrap according to claim 13, wherein said front wrap is oval shaped.

15. The horse ligament wrap according to claim 13, wherein said back wrap is oval shaped.

16. The horse ligament wrap according to claim 13, wherein said front wrap and said back wrap are approximately 26 inches wide by 13 inches long.

17. The horse ligament wrap according to claim 13, wherein said securing tab is disposed on a corner of said horse ligament wrap.

18. The horse ligament wrap according to dam 13, wherein said securing tab is colored and is approximately 5 inches long and said vinyl fabric is approximately 15 inches wide and 12.5 inches long.

19. The horse ligament wrap according to dam 13, wherein said stitching is provided around said perimeter of the ligament wrap.

20. The horse ligament wrap according to claim 13, wherein said horse ligament wrap is used in combination with a liniment to work as a sweat.
